(19) United States
(12) Patent Application Publication
(10) Pub. No.: US 2013/0210016 A1
(43) Pub. Date: Aug. 15, 2013

(54) NUCLEIC ACID DETECTION AND RELATED COMPOSITIONS METHODS AND SYSTEMS

(71) Applicant: Lawrence Livermore National Security, LLC, (US)

(72) Inventors: Lawrence DUGAN, Modesto, CA (US); Brian E. Souza, Livermore, CA (US); Jane P. Bearinger, Berwyn, PA (US)

(73) Assignee: Lawrence Livermore National Security, LLC, Livermore, CA (US)

(21) Appl. No.: 13/767,726

(22) Filed: Feb. 14, 2013

Related U.S. Application Data
(60) Provisional application No. 61/599,225, filed on Feb. 15, 2012.

(51) Int. Cl. C12Q 1/04

(52) U.S. Cl. CPC C12Q 1/04
USPC 435/6.12; 435/287.2

(57) ABSTRACT

Provided herein are methods and systems for loop-mediated isothermal amplification of target polynucleotides on a sample without sample preparation. Methods and systems herein described also allow detection of cells and in particular bacterial cells on an untreated sample comprising the cells, and allow in some embodiments specific detection of bacterial cells such as B. anthracis.

### CROSS-REFERENCE TO RELATED APPLICATIONS

This application claims priority to provisional U.S. application 61/599,225 entitled “Nucleic Acid Detection and Related Compositions Methods and Systems” filed on Feb. 15, 2012 with docket number IL-12518 and is herein incorporated by reference in its entirety.

### STATEMENT OF GOVERNMENT GRANT

The United States Government has rights in this invention pursuant to Contract No. DE-AC52-07NA27344 between the U.S. Department of Energy and Lawrence Livermore National Security, LLC, for the operation of Lawrence Livermore National Security.

### FIELD

The present disclosure relates to nucleic acid detection and to related compositions, methods and systems.

### BACKGROUND

High sensitivity detection of nucleic acid and in particular of biomarkers has been a challenge in the field of biological molecule analysis, in particular when aimed at specific detection of targets such as cells and in particular pathogens. Whether for pathological examination or for fundamental biology studies, several methods are commonly used for the detection of various classes of biomaterials and biomolecules.

Some of the techniques most commonly used in the laboratory for detection of single biological targets have provided the ability to detect one or more biomarkers in biological samples such as tissues and are also suitable for diagnostic purposes.

However, several of the available techniques often require sample treatment or preparation to meet minimal and reproducible assay sensitivity levels.

Therefore, performance of accurate and sensitive nucleic acid detection in a sample remains challenging.

### SUMMARY

Provided herein are methods and systems and related compositions for nucleic acid detection which in several embodiments, allow accurate and sensitive nucleic acid detection in an untreated sample.

According to a first aspect a method and system for detecting a target polynucleotide in an untreated sample, is described. The method comprises: performing loop-mediated isothermal amplification on the untreated sample with primers specific for the target polynucleotide; and detecting amplification of the target polynucleotide following the performing. The system comprises: primers specific for the target polynucleotide and reagents for performing loop-mediated isothermal amplification, for simultaneous combined or sequential use in detecting target polynucleotide in an untreated sample.

According to a second aspect, a method and system to identify a target cell, are described. The method comprises: contacting the target cell with a polymerase and primers specific for a target polynucleotide specific for the target cell for a time under condition to allow performance of loop-mediated isothermal amplification; and detecting polynucleotide amplification following the contacting. The system comprises primers specific for the target cell and reagents for performing loop-mediated isothermal amplification for simultaneous combined or sequential use in detecting target cell in an untreated sample.

According to a third aspect, a method and system to identify Bacillus anthracis in an untreated sample, are described. The method comprise: contacting the untreated sample with a polymerase and primers specific for the Bacillus anthracis for a time under condition to allow performance of loop-mediated isothermal amplification; and detecting polynucleotide amplification following the contacting. The system comprises: primers specific for Bacillus anthracis and reagents for performing loop-mediated isothermal amplification for simultaneous combined or sequential use in detecting Bacillus anthracis in an untreated sample.

Methods and system herein described and related compositions, allow in several embodiments an approach for the rapid detection (e.g. minutes) of nucleic acid compared with existing methodologies and technologies of the art that require time-consuming and laborious methods.

Methods and system herein described and related compositions, allow in several embodiments, accurate and sensitive nucleic acid detection compared with existing methodologies which often generate suboptimal levels of poor quality DNA for analysis and detection.

The methods and systems herein described can be used in connection with medical, pharmaceutical, veterinary applications as well as fundamental biological studies and various applications, identifiable by a skilled person upon reading of the present disclosure, wherein detection of is desirable. Exemplary applications comprise first-responders related applications, decontamination services, diagnostic laboratories, scientific researchers and field veterinarians to test for the presence of organisms.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the detailed description and example sections, serve to explain the principles and implementations of the disclosure.

FIG. 1 shows a gray scale version of a photograph of the colorimetric detection of DNA amplification from unprocessed Bacillus anthracis spores using loop-mediated isothermal amplification (LAMP) and hydroxynaphthol blue. Samples were heated to 63° C. and monitored for color change and in particular light blue shown as light gray for the samples outlined by a black box. Images were captured with a Nikon D90 camera.

FIG. 2 shows graphs of real-time LAMP detection of pag, cap and sap from purified Bacillus anthracis DNA. Purified DNA, 500 pg/reaction was amplified on a BioRad CFX 96 platform using real-time LAMP targeting the pag sequence on pX01, cap on pX02 and sap on the chromosome using previously published primers (Kurosaki et al, 2009). Panel A shows relative fluorescence unit (RFU) traces from instrument. Light grey: pag results, dark grey: cap results, black: sap results. Panel B shows average threshold time in minutes for each target by strain of B. anthracis. Error bars are ±1 SD based on average of three replicate wells. NTC=no template control.

FIG. 3 shows a gel electrophoresis of representative LAMP products. LAMP products were visualized on a 2% agarose gel after 45 min at 63° C. 5 μl of reaction product was loaded per lane.

FIG. 4 shows gray scale version of photographs of culture of samples following amplification and detection wherein the culture is shown as an opaque gray formation on the agar. Following positive amplification, 10 μl of reaction mixture was inoculated onto nutrient agar and incubated overnight at 36° C. Panel A shows post-amplification culture of LAMP reaction containing spores. Panel B shows post-amplification culture of LAMP reaction containing overnight culture.

FIG. 5 shows gray scale versions of photographs of 96-well plates depicting the discrimination of non-pathogenic strains from pathogenic strains. Aqueous suspensions of B. anthracis spores from non-pathogenic, pX01⁺/pX02⁻ Sterne and cells from three pathogenic, pX01⁺/pX02⁺ strains (Ames A0462, Vollum 1B A0488 and PAK1 A0463) were added directly to LAMP reactions targeting pag on pX01, cap on pX02 and the chromosomally-encoded sap gene. Panel A shows a gray scale version of a photograph of a 96-well plate using Sterne spores as template following 60 min amplification at 63° C. which shows positive light blue sample as light gray in the sample within black boxes. Panel B shows a gray scale version of a photograph of a 96-well plate using Ames, Vollum 1B and PAK1 cells as template following 60 min amplification at 63° C. which shows positive samples shown as different shades of gray (original light blue) in the sample within a black box in comparison with negative NTC control (dark blue) shown as black sample in the sample within a dashed box. With reference to the samples within the black box the different shades of gray in the illustration indicate a different degree of positivity with respect to the samples within the dashed box as will be understood by a skilled person also in view of the disclosure.

FIG. 6 shows a gray scale version of a photograph of a 96-well plate depicting the discrimination of non-pathogenic strains from pathogenic strains. Mid-log nutrient broth cultures of B. anthracis from non-pathogenic, pX01⁺/pX02⁻ Sterne UT238 (OD₆₀₀ 0.5) and three pathogenic, pX01⁺/pX02⁺ strains (Ames (OD₆₀₀ 0.8), Vollum 1B (OD₆₀₀ 0.4) and PAK1 (OD₆₀₀ 0.2)) and negative control B. globigii (OD₆₀₀ 0.65) were added directly to LAMP reactions targeting pag on pX01, cap on pX02 and the chromosomally-encoded sap gene. Image of 96-well plate following 30 min amplification at 63° C. NTC: no template control, NB. The positive (light blue) samples are shown in the grayscale illustration as light gray samples within black boxes.

FIG. 7 shows a gray scale photograph with samples from a mid-log culture of Bacillus anthracis cells and the LAMP primers targeting the protective antigen (pag) gene sequence for a 30 minute reaction. 30 CFU per reaction are wells G1, G2, and G3. Negative controls are in well F4-6, G4-6 and H4-6. All other wells are template positive beginning with 3×10⁵ CFU per reaction in wells A1-3 with a 10-fold dilution per row down to 3×10⁻¹ CFU per well in wells B4-6. Wells outlined by a black box indicates positive reaction, and wells outlined by a dashed box indicates negative reaction

FIG. 8 shows a photograph of samples from Clostridium botulinum cells that indicate detection of Botulinum neurotoxin A and B via LAMP. 10-fold dilution series is shown on the right-hand side and negative controls occupy each well of the bottom row. Columns 1-3 and 7-9 represent triplicate samples of cultures grown on agar, while columns 2-4 and 10-12 represent cultures grown in liquid growth medium.

### DETAILED DESCRIPTION

Methods and systems are herein described that allow detection of a target nucleic acid through loop-mediated isothermal amplification.

The term “target nucleic acid” or “target polynucleotide” as used herein indicates an analyte of interest that comprises a nucleic acid. The term “analyte” refers to a substance, compound, moiety, or component whose presence or absence in a sample is to be detected. The term “nucleic acid” or “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term “polynucleotide” includes nucleic acids of any length, and in particular DNA, RNA, analogs and fragments thereof. A polynucleotide of three or more nucleotides is also called “nucleotidic oligomer” or “oligonucleotide.”

Target nucleic acids detectable through the methods and systems herein described include, but are not limited to, biomolecules and in particular biomarkers. The term “biomolecule” as used herein indicates a substance, compound or component associated with a biological environment. The term “biomarker” indicates a biomolecule that is associated with a specific state of a biological environment including but not limited to a phase of cellular cycle, health and disease state. The presence, absence, reduction, upregulation of the biomarker is associated with and is indicative of a particular state. The “biological environment” refers to any biological setting, including, for example, ecosystems, orders, families, genera, species, subspecies, organisms, tissues, cells, viruses, organelles, cellular substructures, plants, animals, amoeba, prions, and samples of biological origin.

In method target nucleic acids can be associated to a cell or viral particles, wherein the cells can be comprised in any organisms including unicellular (consisting of a single cell; including most bacteria) or multicellular (including plants and animals) organisms. Unicellular or multicellular microorganisms and viral particles can be microorganisms.

The term “microorganism” as used herein describes an organism of microscopic or submicroscopic size. Microorganisms are not visible by the naked eye but can be visible under devices such as microscopes and the like. Microorganisms can comprise a single cell, cell clusters, or multicellular complexes. Exemplary microorganisms include viruses, prokaryotes such as bacteria and archaea, eukaryotes such protozoa, fungi, and algae, and animals such as planarians.

In some embodiments of methods and systems herein described, target nucleic acids are detected through Loop-mediated isothermal amplification (LAMP).

The terms “detect” or “detection” as used herein indicates the determination of the existence, presence or fact of a target in a limited portion of space, including but not limited to a sample, a reaction mixture, a molecular complex and a substrate. The terms “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

In particular detection in embodiments of the present disclosure is performed by detecting a signal emitted by a label in one or more amplified target nucleic acid following Loop-mediated isothermal amplification

The wording “Loop-mediated isothermal amplification,” or “LAMP” as used herein indicate a technique capable of rapidly amplifying specific nucleic acid sequences without specialized thermal cycling equipment. In particular, Loop-mediated isothermal amplification (LAMP) indicates an isothermal nucleic acid amplification technique such as the one originally described by Notomi and coworkers in 2000 (Notomi 2000). In some embodiments, the LAMP technique utilizes four primers that target six distinct sequences on the template nucleic acid. The addition of reverse transcriptase into the reaction, termed reverse-transcription LAMP or RT-LAMP, allows for the detection of RNA templates under the same conditions (Notomi 2000). The addition of loop primers was subsequently shown to increase the rate of the reaction, reducing overall amplification times significantly (Nagamine 2002). LAMP in the sense of the present disclosure comprise LAMP reaction and RT-LAMP such as the ones reported for numerous human and animal bacterial, protozoan and viral pathogens including, for example, Salmonella enterica (Ohtsuka 2005), African trypanosomes (Kuboki 2003) and Foot and Mouth disease virus (Dukes 2006), amongst many others. LAMP in the sense of the present disclosure also comprise LAMP assays for the detection of B. anthracis such as the ones described in Qiao 2007, Kurosaki 2009, Hatano 2010, and Jain 2011. LAMP in the sense of the present disclosure further comprises detection techniques in which DNA isolated from spores spiked into soil and talcum powder is detected by LAMP targeting the pag gene on pX01 such as the one described in Jain 2011.

In methods and systems herein described, LAMP can be performed with primers that are specific for the one or more target polynucleotide to be identified.

The wording “specific”, “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred. The term “specific” with reference to two items indicate a peculiar association between the items so that first item is uniquely associated with the second item that therefore uniquely identifies and sets apart an item discriminating among similar, and is not limited to simple detection (e.g. a target polynucleotide specific for a cell or group of cells is a polynucleotide that is uniquely associated with that cell and can be used therefore to identify the cell among others).

The term “primer” as used herein refers to an oligonucleotide capable of binding to a particular region of a target polynucleotide and serves as a starting point for the amplification reactions that occur during the LAMP reaction at the appropriate temperatures described herein, and in the presence of the appropriate DNA polymerase and/or reverse transcriptase, that permit the amplification and the resulting detection of the target polynucleotide. Suitable primers for the LAMP reactions can be designed, for example, by inputting the nucleotide sequence of the target polynucleotide into software such as PrimerExplorer (primerexplorer.jp/e/index.html), Primer3 (frodo.wi.mit.edu/), and others identifiable by a skilled person. A skilled person, upon a reading of the present disclosure can design primers with specificity to a desired target polynucleotide in order to minimize false positive results. In particular, primers can be designed for optimal binding to target nucleic acid at the enzyme functional temperature as will be understood by a skilled person.

In some embodiments, methods and systems herein described can comprise the use of multiple primer sets each specific for a target polynucleotide. In some of those embodiments, the one or more target polynucleotides can be specific for a cell (e.g. a bacterial cell) and can be used to detect said cell. By way of example, the use of three primer sets targeting both plasmids and the chromosome of B. anthracis allows for the rapid discrimination of non-pathogenic bacteria from pathogenic bacteria within minutes of sampling (see, for example, Examples 1, 3-7, and 9-10). Additional primer sets directed to identify other bacterial cells, or cells can be identified by the skilled person upon reading of the present disclosure. Multiple primer sets can be designed and evaluated for each target nucleic acid to be detected.

In some embodiments, the methods and systems herein described allow detection of one or more target polynucleotides in an untreated sample, without the need of having sample preparation directed to nucleic acid isolation.

The term “sample” as used herein indicates a limited quantity of something that is indicative of a larger quantity of that something, including but not limited to fluids from a biological environment, specimen, cultures, tissues, commercial recombinant proteins, synthetic compounds or portions thereof, aqueous suspensions of spores, liquid bacterial cultures, and bacterial cultures grown on solid agar. Additional samples can include, but are not limited to, environmental samples, such as water from various sources, soil and aerosols collected via aerosol collection devices; animal samples, such as body fluids, tissue, and wound exudite; environmental, laboratory and clinical samples containing bacterial cells and spores, virus particles, fungi, or mammalian cells; and human samples, such as body fluids, tissue, urine, decanted feces, whole blood and blood culture media and wound exudite.

The term “sample preparation” indicates techniques and procedures directed to increase availability of a target analyte in the sample. In most analytical techniques known in the art, sample preparation is a very important step because the techniques are often not responsive to the analyte in its in-situ form, or the results are distorted by interfering species. Exemplary sample preparation procedures that according to embodiments herein described are not performed prior to initiating LAMP comprise forms of sample nucleic acid extraction and isolation such as boiling, chemical and/or mechanical lysis of a sample, nucleic acid purification, centrifugation of a sample and additional techniques performed on samples that are identifiable by a skilled person. In particular, in methods and systems herein described LAMP is therefore performed on untreated samples.

The term “untreated sample” as used herein indicates to a sample that has not been subjected to sample preparation, wherein sample preparation refers to the ways in which a sample is treated prior to its analysis to increase availability of the target of the analysis. Exemplary sample preparation directed to increase availability of a target nucleic acid comprise treatment directed at lysing cells and, in particular, extracting and/or isolating DNA, RNA, gene sequence, or other target polynucleotides and additional techniques performed on the sample prior to LAMP amplification.

In particular, in several embodiments herein described, sample preparation refers to techniques directed to treat, extract and/or isolate polynucleotides and in particular the target polynucleotides from the sample. More particularly sample preparation or treatment to isolate nucleic acid comprises chemical, mechanical or physical isolation of the nucleic acid from the sample as well as other procedures directed to increase availability of a target polynucleotide identifiable by a skilled person.

In some embodiments, “untreated samples” can be subjected prior to use in the methods and systems herein described to techniques directed to increase detection of the target polynucleotide available in the untreated sample, which are in particular performed without breaking open a biological material possibly present in the sample, and/or resulting in purified nucleic acid free of other cellular components also possibly present in the sample. More particularly, techniques can be used to increase compatibility and/or suitability of the sample for a LAMP reaction of choice which in turn provides a detectable amplified product according to the present disclosure. Exemplary techniques to increase the physical and/or chemical compatibility and/or suitability of a sample to a LAMP reaction comprise dissolution in a suitable solvent, reaction of the sample with some chemical species directed to allow formation of a LAMP reaction mixture, pulverizing, treatment with a chelating agent (e.g. EDTA), masking, filtering, concentration, dilution, sub-sampling, slurry formation, or other techniques identifiable by a skilled person upon reading of the present disclosure. Selection of a suitable technique to increase compatibility and/or suitability of a sample to a LAMP reaction of choice can be performed by a skilled person upon reading of the present disclosure based on the chemical and physical status of the untreated sample and can be in particular directed to obtain an untreated sample in a form increasingly suitable to allow performance of a LAMP reaction. For example, in embodiments where the untreated sample comprises dirt or pulverized material, addition of an aqueous solvent can be performed to obtain a slurry of various suitable densities and consistencies to which LAMP reaction reagents are then added. In other exemplary embodiments where the untreated sample is large volume of liquid, a centrifugal evaporator or vacuum concentrator can be performed to decrease the total volume of the liquid to be compatible with LAMP reaction reagents. In embodiments where the untreated sample is a mixture of solid and liquid components, for example, separation of components with woven wire cloth can be performed to split the untreated sample into the untreated and filtered solid and liquid components. In those embodiments once separated into the untreated components, a skilled person can use suitable techniques to increase the physical and/or chemical compatibility or suitability of the individual components to LAMP reaction.

Exemplary untreated samples that can be subjected or not to additional treatment to increase detection of the target nucleic acid comprise samples wherein the target nucleic acid has not been subjected to mechanical or physical isolation from a cell or viral particle. Mechanical or physical isolation of nucleic acids from cells or cell or viral particle, can be performed by cell lysis, cell homogenization, cells sonication, glass bead treatment of cells, French press of cells and additional techniques identifiable by a skilled person.

Accordingly in some embodiments herein described the amplification of nucleic acid and the detection of the amplification product can be performed without mechanical or physical isolation of the nucleic acid from the sample prior to addition to the LAMP reaction. For example, in some embodiments, an untreated sample is an aqueous solution and a skilled person would add the aqueous solution to the LAMP reaction without additional steps to isolate nucleic acid. In another embodiment, for example, an untreated sample is a powder, and a skilled person would resuspend the powder in a solution compatible with LAMP to increase detection without additional steps to isolate nucleic acid. Also, for example, in some embodiments, an untreated sample can comprise a culture of cells and a skilled person can add the cells directly to the LAMP reaction mixture without additional steps to isolate nucleic acid.

In some embodiments, availability of the target nucleotide in an untreated sample can be increased during the LAMP reaction according to methods and systems herein described. In particular, lytic enzymes and/or other suitable reagents can be added to the LAMP reaction mixture to lyse cells comprising a target polynucleotide or otherwise increase availability of the target polynucleotide by performing reactions occurring concurrently with the performance of the LAMP reaction. In addition or in the alternative, in some embodiments the reaction conditions of the LAMP reactions can be adjusted to increase availability of the target polynucleotide during performance of the LAMP reaction.

Selection of the appropriate enzyme and reaction conditions can be performed by a skilled person in view of the specific target polynucleotide to be detected the known or expected location with respect to a related cell or viral particle also expected to be present in the untreated sample as well as in view of the desired experimental design as will be understood by a skilled person. In particular, selection of the lytic enzyme or other reagents as well as timing of the addition of a lytic enzyme or other reagent, and selection of appropriate reaction condition suitable to increase availability of the target polynucleotide during LAMP can be performed based on the designed LAMP reaction, related reagents and conditions. More particular, lytic enzyme and other reagents can be selected to be active at temperature and conditions compatible with the LAMP reaction of choice and be directed to allow a lytic reaction to occur at some time during said LAMP reaction. For example, in some embodiments, lysozyme, can be selected for use during LAMP reaction. Lysozyme is stable and active between pH 4 and 5 and up to 63° C., and acid solutions of lysozymes are stable even at temperatures up to 100° C., compatible to be concurrently active with exemplary LAMP reaction conditions. In another exemplary embodiments, proteinase K, can be used for use during LAMP reaction. Proteinase K is a broad-spectrum serine protease, is stable and active from temperatures from 50-65° C. and a pH range of 4-12, also compatible with exemplary LAMP reaction conditions. Also reaction conditions of the LAMP can be adjusted to increase availability of the target polynucleotide during LAMP reaction according to embodiments of methods and systems herein described. For example, the pH of LAMP reactions can be adjusted to increase the activity of lytic enzymes at a value that still fall in the active pH range of LAMP enzymes. Chemicals such as of sodium dodecyl sulfate (SDS), Guanidinium chloride, or Guanidinium thiocyanate can also be added to increase the activity of a lytic enzyme such as Proteinase K but in small enough concentrations as to not completely inhibit the LAMP reaction in accordance with the experimental design as will be understood by a skilled person.

In general, embodiments where addition of lytic enzymes or other suitable reagents is performed in connection with the LAMP reaction, are directed to increase availability of the target polynucleotide during the LAMP reaction. In particular, some of those embodiments are performed to increase availability of target polynucleotide known or expected to be located inside a microorganism, (e.g. fungi), or cells such as mammalian cells and tissue. In some of those embodiments a lytic reagent can be added in very small quantities to the LAMP reaction mixture and the lysis be performed simultaneously to the LAMP reaction or in a short, (e.g. <10 min), step added at the beginning of the LAMP reaction. Exemplary embodiments wherein use of lytic enzymes or other suitable reagents can be desired comprise embodiments wherein the target nucleotide is comprised within cells or microorganisms that do not readily shed nucleic acid or which have sufficient outer walls or membranes that impair the uptake of the LAMP reaction components to the extent necessary to perform the reaction.

For example, in some embodiments, methods herein described allow LAMP detection of a target nucleic acid which is comprised in the untreated sample within microorganisms such as viral particles, cells, and/or spores or cell from a pluricellular organism (e.g. animal or plants). In particular, in some embodiments the target nucleic acid can be comprised within cells, such as cell lines or other cells, and cellular microorganisms such as bacteria. In some of those embodiments, selection of the appropriate reaction conditions can be performed based on the features of the microorganism at issue and in particular on the temperature resistance of the microorganism at the temperature of the LAMP reaction. For example, in some embodiments, the microorganism is E. coli which undergoes lysis from temperatures 42° C. and above allowing the release of target nucleic acid at exemplary LAMP temperatures. In other embodiments, for example, the microorganism is Bacillus psychrophilus which undergoes lysis from temperatures 37° C. and above allowing the release of target nucleic acid. In another embodiment, for example, the microorganism is Bacillus anthracis, which undergoes heat shock and subsequently releases nucleic acid at temperature range of 60-70° C. In particular, in embodiments, wherein the target polynucleotide is expected to the located within a microorganism that is temperature resistant at the LAMP reaction conditions one or more lyric enzymes or other suitable reagents can be performed to increase the availability of the target polynucleotide at issue. In some embodiments, for example, the microorganism is a thermophile such as Thermus aquaticus or Bacillus stearothermophilus, which thrive at temperatures above some exemplary LAMP reactions, and a skilled person may use a lytic enzyme, such as lysozyme, to facilitate release of nucleic acid.

In some embodiments, the untreated sample comprises target nucleic acid comprised outside of microorganisms such viral particles, cells and spores, and/or outside cells from a multicellular organisms. In particular, in some embodiments, methods herein described allow LAMP detection of a target nucleic acid comprised outside viral particles, cells, and/or spores after lysis of the viral particles, cells, and/or spores in the untreated sample performed during LAMP reaction. In some embodiments, the detected target nucleic acid of the untreated sample is possible present, for example, in or on dust particles, protein aggregates, clothing, and other surfaces identifiable to a skilled person upon a reading of the present disclosure. In those embodiments a target nucleic acid can be detected using the method described within following a simple collection protocol and introduction into a reaction mixture. Exemplary samples wherein methods and systems herein described can be performed to detect target nucleic acid available for LAMP detection in the sample free of cell component comprise soil, aerosols, dust particles, protein aggregates, clothing and other surfaces, human and animal blood, saliva, feces, urine, stomach acids, boils, blisters, open sores and wounds, fluids found in plant roots and stalks.

In some embodiments, the viral particles, cells, and/or spores known or expected to be located inside or outside of which the target polynucleotides are detectable by the methods and systems herein described can include, but are not limited to, human papillomavirus (Luo 2011), human enterovirus 71 and Coxsackievirus A16 (Nie 2011), African trypanosomiasis (Wastling 2010), turkey coronavirus (Cardosa 2010) and H1N1 influenza (Ma 2009), Salmonella enterica (Ohtsuka 2005), African trypanosomes (Kuboki 2003) and Foot and Mouth disease virus (Dukes 2006), B. anthracis (Qiao 2007, Kurosaki 2009, Hatano 2010, Jain 2011), and others identifiable to a skilled person upon reading of the present disclosure. In embodiments in which the target polynucleotide are from viruses, a skilled person, typically, adapts addition of lytic enzymes to LAMP reaction based on the infected cells resistance to heat shock or lysis at LAMP temperatures.

In methods and systems herein described, target polynucleotides can have various lengths compatible with the LAMP reactions which are identifiable by a skilled person. For example in some embodiments, the target can be a polynucleotide from approximately 50 to approximately 500 nucleotides in length. In methods and systems herein described the target polynucleotide can be of different kinds and originated from various sources. For example target nucleic acid can comprise DNA, RNA or Peptide nucleic acid (PNA) and additional nucleic acid identifiable by a skilled person. In some embodiments, the target nucleic acid can be originated from natural sources or being synthetic. For example, the target nucleotide can be a gene sequence or a regulatory sequence. In some embodiments, the target polynucleotide can be a biomarker. The term “biomarker” as used herein indicates a substance or characteristic used as an indicator of the presence of a biological state or material, such as a phase of cellular cycle, a biological process, as positive indication of a molecule or organism. Typically, presence, absence, reduction, upregulation of the biomarker is associated with and is indicative of a particular state, such as an active or inactive form of a microorganism. In some embodiments, the polynucleotide can be synthetic and in particular. In embodiments, where the target is RNA, reverse transcriptase enzyme is typically added to the reaction to convert the RNA to DNA.

In some embodiments, the target polynucleotide is the gene coding for the S-associated protein (sap) which can be used to identify B. anthracis cells. Additional target polynucleotides specific to B. anthracis can be used with the methods and systems herein described and include, but are not limited to, cya, lef, pagA, capA, capB, capC, and others identifiable to a skilled person upon reading of the present disclosure.

In an exemplary embodiment, appropriate primers for a target nucleic acid of interest can be used in addition to an appropriate LAMP detection reagent (such as, for example, hydroxynaphthol blue) and other LAMP reagents identifiable to a skilled person to create a LAMP mixture (as described, for example, in Notomi 2000 or in the Examples section of the present disclosure). The LAMP reagents and the untreated sample can be combined in any order and the reaction conditions can be set according to the desired experimental design identifiable by a skilled person. In particular, in embodiments, the LAMP reagents are first mixed to obtain a LAMP reagents mixture then added to the untreated sample to provide a LAMP reaction mixture. In some embodiments, the LAMP reagents mixture does not comprise the LAMP enzyme which is added to the mixture following addition of the untreated sample to provide the LAMP reaction mixture. In some embodiments, a lytic enzyme or other reagents suitable to increase availability of the target nucleic acid can be added to the LAMP reagents mixture or to the LAMP reaction mixture possibly concurrently with the addition of the LAMP enzyme.

An untreated sample can be contacted with the LAMP mixture (such as, for example, by adding the untreated sample directly to the LAMP mixture or by adding the LAMP mixture directly to the untreated sample) and the LAMP mixture is heated to an appropriate temperature to allow the LAMP reaction to occur. Detection of a possible amplified target polynucleotide can then be performed by detecting labels present in the LAMP reaction mixture directly on the mixture or possibly following transferring of the mixture on a suitable support (e.g. gel followed by ethidium bromide staining).

The terms “label”, “labeled molecule” as used herein as a component of a complex or molecule refer to a molecule capable of detection, including but not limited to molecules emitting a labeling signal and molecules capable of binding with a compound emitting a labeling signal (e.g. through a functional group capable of reacting with a corresponding functional group on the compound emitting the signal). Exemplary molecules capable of direct detection comprise as radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting detectable fluorescence. As a consequence, the wording “labeling signal” as used herein indicates a detectable signal that allows detection of the label, including but not limited to radioactivity, fluorescence, chemiluminescence, production of a compound in outcome of an enzymatic reaction and the like. In some embodiments the labeling signal is emitted directly from the label, in some embodiments the labeling signal is emitted from a compound attached to the label.

For example in some embodiments, following successful amplification of the target nucleic acid a LAMP detection reagent can be detected by suitable techniques such as, by a color change in the heated LAMP mixture if a colorimetric detection reagent is used. The amount of time necessary for detection would be identifiable to a skilled person upon reading of the present disclosure and is a function of factors such as, for example, detection method (for example, turbidity or colorimetric detection or fluorescent detection) the quantity of sample, the number of copies of the target sequence, and the quality of the primer design and the quality of the primer synthesis/purification. In some embodiments, the amount of amplification product can be quantified post-detection using spectrophotometry (e.g. UV/visible spectrophotometer) or fluorometry (e.g. Qbit plus standard DNA curve).

In some embodiments, methods and systems herein described can be directed to detect multiple target polynucleotides in the untreated sample. In some of those embodiments, a quencher labeled “inner primer” can be annealed for example to a labeled complimentary sequence; upon incorporation of the inner primer into the amplification product, the product can be detected qualitatively e.g. by turbidity or a colorimetric, fluorescent, or bioluminescent label incorporated into the reaction (Tanner 2012). The number of targets in a LAMP reaction is based on the target sequences and the design of primers used in the simultaneous LAMP reactions. A skilled person has software tools such as MPrimer or PrimerPlex to design multiple primers that with anneal to targets without interference.

In methods and systems, LAMP can be performed at any suitable LAMP reaction temperature identifiable by a skilled person. In some embodiments, the LAMP reactions are performed at a temperature of from about 60 to about 65° C. The skilled person will adjust the temperature of the reaction based on the optimal temperature for the enzymes used in the LAMP reaction. The temperature can be maintained by means such as a water bath, a heating block, a thermal cycler, an oven, or another means identifiable to a skilled person as being capable of maintaining the desired temperature range over the course of the reaction.

In some embodiments, LAMP reagents are prepared ahead of time, omitting the LAMP enzyme e.g. Bst polymerase. Reaction mixtures absent polymerase and template can be stable for weeks or longer at 4° C. The stability of LAMP reaction mixtures containing fluorescent detection dyes may be affected by exposure to light. Bst polymerase is added to the LAMP reaction immediately prior to adding the sample. The sample is added last to the reaction chamber (tube, 96-well plate, other) containing the proper volume of LAMP reaction solution.

In some embodiments, it is expected that a lytic agent that will lyse cells can be added to the LAMP reaction solution before or after addition of the untreated sample to the LAMP reaction mixture, thus freeing the target nucleic acid for amplification and detection. Other researchers (Liu et al., 2009, J Clin Virol (46) p. 49-54) have shown that it is possible to fix virus-infected mammalian cells with paraformaldehyde, permeabilize the cell membrane and perform LAMP reactions on viral nucleic acid. Suitable lytic agents include, but are not limited to, lysozymes, muramidases, endolysins or other lysing agents identifiable to a skilled person upon reading of the present disclosure. Lytic reagents may be used, for example, in the LAMP reaction mixtures when the sample is resistant to release of nucleic acid at the LAMP reaction temperatures such as, but not limited to, thermophiles.

In some embodiments, LAMP amplification products from the untreated samples can be used for further downstream applications such as, for example, sequencing or genotyping. For example in some embodiments, sequencing can be performed following an approach similar to sequencing a PCR reaction product using a commercial kit and instrumentation commercially available (e.g. from ABI and other vendors) as will be understood by a skilled person. In some of those embodiments, due to the nature of the LAMP product, repeating inversions of the target sequence on a limited number of strands of DNA product, incorporation of restriction enzyme cut sites in the product can improve sequencing results. In some embodiments, LAMP product can be digested by restriction enzyme prior to the sequence labeling reaction.

Further embodiments are described in the following in which methods and systems of this disclosure are described in connection with exemplary cell or viral particle, target polynucleotides, reactions conditions, detection techniques and possible further analysis and additional parameters of the LAMP reaction performed according to method and systems herein described. A skilled person will be able to adapt the relevant description to additional detection techniques, microorganisms, target polynucleotides, reactions conditions, further analysis and additional parameters of the LAMP reaction in view of the present disclosure.

In particular in some embodiments, methods and systems herein described can enable rapid nucleic acid amplification via Loop-mediated isothermal amplification (LAMP) within minutes of sample addition without sample processing. In particular, Applicants have demonstrated detection of pathogenic Bacillus anthracis cells in under 15 minutes and spores of Bacillus anthracis in under 25 minutes (see e.g. Example 7). Detection time may increase slightly with decreasing initial concentration of target. The amount of target polynucleotide and the means of detection affect the timing of positive detection, e.g. high concentrations of target polynucleotide can inhibit the speed of positive detection and fluorescence as detected by a real-time device can confirm positive detection faster than turbidity or colorimetry.

In a particular example, Applicants were able to colorimetrically detect 10-100 colony forming units (CFU) of B. anthracis Sterne spores in 20-30 minutes and 1-10 CFU in 30-40 minutes by adding the untreated samples directly to LAMP reaction mixtures and using primers specific to the pag gene (see, for example, Example 7). In addition to colorimetric assays, skilled person may use a fluorescent indicator in conjunction with LAMP (see, for example, Example 2). Colorimetric and fluorescent indicators used in LAMP to generally give a positive or negative result can also be measured for absorbance to quantify LAMP amplified products post reaction. In addition, real-time devices used with fluorescence can provide data regarding quantification as the reaction proceeds.

In some embodiments, for example reactions using spores as template can be plated onto nutrient agar and successfully expanded, allowing for further assaying of the bacteria, if desired (see, for example, Example 8). In view of results illustrated in the Examples section, near real-time amplification, detection and discrimination of non-pathogenic from pathogenic samples is expected for “crude samples” of cells and in particular bacterial cells such as B. anthracis. In particular, in some embodiments wherein the untreated sample is a crude sample, the untreated sample is expected to possibly comprise actively dividing mid-log cultures, quiescent cultures and spores. In several embodiments, the elimination of several sample-processing steps (sample lysis, DNA purification, centrifugation, etc.) is expected to reduce the cost and time required to detect target nucleic acid and related cells such as B. anthracis DNA from samples that contain spores or from liquid fermentations containing spores or vegetative cells.

In some embodiments, methods and systems herein described comprise performing LAMP from vegetative cells and spores (see Examples section with reference to Bacillus anthracis and in particular Example 3) without nucleic acid extraction. In some embodiments, the simple addition of cells or spores to the reaction mixture, followed by heating at a temperature that is compatible for LAMP reaction is all that is required to reproducibly amplify and detect target plasmid and chromosomal DNA via colorimetric change or other detection methods identifiable by a skilled person upon reading of the present disclosure.

In some embodiments, a sample possibly containing viral particles, cells or spores is simply added to the reaction mixture, heated to a temperature compatible with LAMP and amplification is detected via colorimetric change, fluorescence and/or other detection methods identifiable to a skilled person upon reading of the present disclosure. In some embodiments, colorimetric and turbidity changes can be detected, for example, by eye or through devices such as a turbidometer and spectrophotometer. Fluorescence can be detected, for example with a UV lamp, a fluorescence detector, or a combination of excitation LEDs and emission silicon photodiodes. Another example of detection known to the skilled person is running the amplification product from a LAMP on an agarose gel or capillary gel system.

In methods and systems herein described, detection of amplification can be performed following the LAMP amplification, using various detection methods applicable in connection with LAMP amplification and identifiable by a skilled person. Suitable methods to detect amplification including turbidity, fluorescence and gel electrophoresis, have been reported for identifying the amplification products of LAMP (reviewed in Parida 2008). Simple colorimetric detection of LAMP reaction products using hydroxynaphthol blue (HNB) was recently described (Goto et al. 2009) and subsequently utilized by other research groups to identify human papillomavirus (Luo 2011), human enterovirus 71 and Coxsackievirus A16 (Nie 2011), African trypanosomiasis (Wastling 2010), turkey coronavirus (Cardosa 2010) and H1N1 influenza (Ma 2009). HNB undergoes a color change as pH and/or cation levels change (Brittain 1978). LAMP reactions generate a significant amount of pyrophosphate byproduct as the amplification product is formed. The excess pyrophosphate bonds with Mg²⁺ in the LAMP reaction, thereby reducing the cation level, resulting in the reaction mixture changing from a violet to a sky-blue color easily detectable with the human eye. Wastling and coworkers found this color change to be superior for visually detecting positive LAMP reactions compared to the orange-to-yellow change from post-reaction addition of Quant-iT Picogreen or the faint orange to green color change detected using calcein with MnCl₂ in the reaction (Wastling 2010). The HNB visual detection method was recently combined with a low-cost disposable sample preparation device to allow for all-in-one sample collection, preparation, amplification and detection of bacterial DNA and viral RNA (Bearinger 2011).

In some embodiments, methods and systems herein described are performed on cell and in particular or cellular microorganisms. In some of those embodiments, methods and systems herein described can be performed on bacterial cell such as B. anthracis. Bacillus anthracis (B. anthracis) is a gram-positive rod-shaped bacterium normally found as spores in soil. These spores are somewhat resistant to pH extremes, desiccation, heat and chemicals. Exposure to virulent spores through inhalation, ingestion or abraded dermal contact results in spore germination and outgrowth into vegetative cells, resulting in anthrax disease in susceptible mammals, particularly herbivores, and humans. Host death usually occurs following bacteremia and subsequent release of the tripartite anthrax protein toxin consisting of edema factor (EF), lethal factor (LF) and protective antigen (PA) (Inglesby 2002 JAMA). B. anthracis has been considered an ideal biological warfare agent due to its stability and infectivity as a spore (Pile 1998) and it was used in acts of bioterrorism in Japan in 1993 by the Aum Shinrikyo cult (Keim 2001) and in the United States in 2001 (Jernigan 2001). The genome of the pathogenic forms of B. anthracis includes a 5.23 Megabase chromosome (Read 2003) and two large plasmids, pX01 and pX02. The pX01 plasmid encodes virulence genes for the anthrax toxin complex involving the proteins edema factor, lethal factor and protective antigen encoded by cya, lef, and pagA, respectively. Plasmid pX02 encodes three capsule synthesis genes capB, capC and capA that are required to produce a poly-γ-D-glutamic acid capsule (Okinaka 1999). Non-pathogenic strains lack one or both of these plasmids (Mikesell 1983).

In some embodiments, the methods and systems herein described permit detection of Bacillus anthracis using LAMP on untreated samples without the need of isolated DNA as a template, whether extracted using phenol/chloroform (Hatano 2010), commercial kits (Kurosaki 2009) or boiling spores (Qiao 2007, Kurosaki 2009, Jain 2011). (see, for example, Examples L 3-7, and 9-10) In particular, in several embodiments, methods and systems herein described allow performance of LAMP without the need of procedures that at times can produce quality DNA preparations suitable for PCR and LAMP, but can require a minimum of 1 hour to perform and laboratory equipment such as tabletop centrifuges capable of speeds>10K RPM.

In some embodiments, detection, and in particular colorimetric detection, of LAMP reactions with B. anthracis spore and cell culture samples, can be performed through direct sampling of active cultures or spores allowing qualitative detection in tubes array plates or similar platforms. In some embodiments, the methodology adds samples directly, whether cells or spores, to the LAMP reaction mixture, which is then heated to a temperature compatible with LAMP reaction. Positive detection of target polynucleotide such as, pag, cap, and sap, was possible in as early as about 10-20 minutes using cells of pathogenic, pX01+/pX02+ strains of B. anthracis (Ames, Vollum 1B, PAK1) as template. Positive detection of pag and sap can be possible in as early as 20-25 minutes using cells and spores from a non-pathogenic, pX01+/pX02− strain of B. anthracis (Sterne) as template. In additional embodiments, lytic enzymes, including lysozymes, muramidases, endolysins or others identifiable to a skilled person upon reading of the present disclosure can be added to the LAMP reaction to improve cell lysis and decrease time to positive amplification detection.

In an exemplary embodiment, an untreated sample of B. anthracis cells (for example, from a broth) is added directly to a LAMP mixture containing primers specific to target polynucleotides of B. anthracis cells (such as, for example, pag, cap, and/or sap) together with a detection reagent (such as, for example, hydroxynaphthol blue) to detect the LAMP reaction products indicating the presence of B. anthracis cells (see, for example, Examples 3-7 and 9-10). The methods and systems exemplified by the aforementioned exemplary embodiment can be applied to detect the presence of B. anthracis cells in untreated test samples (such as, for example, soil samples or water samples). Additional microorganisms and their respective target nucleotides and target polynucleotide primers would be identifiable to a skilled person upon reading of the present disclosure.

In some embodiments, methods and systems herein described allow colorimetric detection of LAMP reactions with B. anthracis spores and vegetative cell culture samples without the need for sample preparation. Spores or cells are simply added to the LAMP reaction mixture, heated to a suitable temperature and positive amplification is detected via color change from purple to blue. In addition, the inclusion of protease inhibitors in the LAMP reaction resulted in an approximately 10-fold increase in sensitivity.

In some embodiments, the methods and systems described herein permit differentiation between pathogenic and non-pathogenic microorganisms e.g. by performing the LAMP reaction on untreated samples using primers for target polynucleotides specific to either the pathogenic or non-pathogenic microorganisms. In a particular example, non-pathogenic spores and cells of B. anthracis were differentiated from pathogenic spores and cells in under 30 minutes by adding the untreated samples of spores or cells directly to a LAMP reaction mixture containing specific primers for the pag, cap, and sap target polynucleotides (see, for example, Examples 9 and 10). Additional target polynucleotides for differentiating between pathogenic and non-pathogenic forms of microorganisms using the methods and systems described herein would be identifiable to the skilled person.

Embodiments, of the methods and systems herein described allow detection of Bacillus anthracis using LAMP on untreated samples without the need of isolated DNA as template, whether extracted using phenol/chloroform (Hatano 2010), commercial kits (Kurosaki 2009) or boiling spores (Qiao 2007, Kurosaki 2009, Jain 2011). In particular, in several embodiments, methods and systems herein described allow performance of LAMP without the need of procedures that at times can produce quality DNA preparations suitable for PCR and LAMP, but require a minimum of 1 hour to perform and laboratory equipment such as tabletop centrifuges capable of speeds>10K RPM.

In some embodiments, methods and systems herein described allow colorimetric detection of LAMP reactions with Bacillus anthracis spores and vegetative cell culture samples without the need for sample preparation. Spores or cells are simply added to the LAMP reaction mixture, heated to a suitable temperature and positive amplification is detected via color change from purple to blue. In addition, the inclusion of protease inhibitors in the LAMP reaction resulted in a ˜1-log increase in sensitivity. The addition of lytic enzymes, including lysozymes, muramidases or endolysins may be added to improve cell lysis and decrease time to positive amplification detection. Samples may include environmental, laboratory and clinical samples containing bacterial cells and spores, virus particles, fungi, or mammalian cells.

In some embodiments, the methods and systems herein described allow detection of target polynucleotide at a concentration lower than or equal to about 10 fg per reaction, and spore detection with a limit lower than or equal to about 10 spores. Qiao and coworkers originally reported detection of three gene targets representing the B. anthracis plasmids, pX01 (pag) and pXO2 (capB), along with a chromosome target (Ba813) using LAMP, with a lower limit of detection of 10 spores (Qiao 2007) using fluorescence and gel electrophoresis. Kurosaki et al. reported detection of three B. anthracis target genes (pag, capB, and sap) again representing the plasmids and chromosome, respectively, with a limit of detection for pag of 10 fg per reaction in approximately 30 min using purified DNA and real-time turbidity detection (Kurosaki et al., 2009). Additionally, Kurosaki et al. reported detecting target DNA from spores isolated from blood of intranasally infected mice (Kurosaki 2009). Hatano and coworkers reported detecting 1000 copies of pag and capB target DNA using LAMP along with a low-cost pocket warmer as a heat source (Hatano 2010).

In some embodiments, detection and in particular colorimetric detection of LAMP reactions with B. anthracis spore and cell culture samples through direct sampling of active cultures or spores. In some embodiments, the methodology adds samples directly, whether cells or spores, to the LAMP reaction mixture, which is then heated to a temperature compatible with LAMP reaction. Positive detection of target polynucleotide such as, pag, cap, and sap, was possible in as early as about 10-20 minutes using cells of pathogenic, pX01+/pX02+ strains of B. anthracis (Ames, Vollum 1B, PAK1) as template. Positive detection of pag and sap was possible in as early as 20-25 minutes using cells and spores from a non-pathogenic, pX01+/pX02− strain of B. anthracis (Sterne) as template.

Published results indicate that LAMP reactions are less susceptible to inhibitors of PCR reactions, such as urine, decanted feces, whole blood and blood culture media (Francois 2011). Methods and systems herein described, in some embodiments allow direct testing of aqueous suspensions of B. anthracis spores and vegetative cells in solid and liquid culture using the LAMP assay combined with colorimetric detection and are expected to greatly simplify and shorten the detection process by eliminating nucleic acid purification. Freshly prepared spores, partially germinated spores, colonies from agar dishes, overnight cultures, cells of early- and mid-log cultures of the Sterne strain along with overnight cultures, cells of early- and mid-log cultures of three pathogenic strains of B. anthracis have been successfully amplified following this protocol. Additionally, protease inhibitors added to the LAMP reaction are expected to improve lower limits of detection. Additionally, testing samples with a combination of primer sets for targets on both B. anthracis plasmids and the chromosome may permit identification of non-pathogenic and pathogenic B. anthracis within 30 minutes, while allowing for the direct re-culturing of positive samples for additional testing and verification.

In some embodiments, methods and systems herein described allow a rapid detection (e.g. minutes) of nucleic acid that is a significant improvement over certain existing methodologies and technologies that are current state-of-the art which often generate suboptimal levels of poor quality DNA for analysis and detection. Some of these embodiments do not require nucleic acid isolation and purification that can involve enzymatic and/or physical breaking of cells followed by removal of proteins and non-target nucleic acid, a process that can require an additional hour to overnight incubation to complete.

In some embodiments, the target polynucleotide resides in microorganisms that undergo partial to complete lysis at LAMP reaction temperatures, e.g. Bacillus psychrophilus can lyse when heated at 37 degrees (Mattingly, 1971), Escherichia coli K12 can lyse when heated at 42 degrees (Membrillo-Hernandez, 1995), and protoplasts of Sarcina lutea and Streptococcus faecalis can undergo thermal lysis when heated to 60 degrees (Ray, 1971) which are all temperatures below an exemplary LAMP reaction temperature of 60-65 degrees. Temperatures higher than optimal growth temperature of a cell can enlarge pores in cell membranes, induce expression of autolysins, and/or melt lipid cell structures allowing release of nucleic acid into a LAMP reaction. In some embodiments, the target polynucleotide resides in microorganisms that undergo partial to complete lysis at LAMP reaction salt conditions, e.g. Clostridium saccharoperbutylacetonicum can be lysed by sodium ion concentrations above 0.1 M (Ogata, 1974). Variations in salt concentrations can induce osmotic lysis in cells which allow release of nucleic acid into a LAMP reaction mixture. Lytic reagents may be used, for example, in the LAMP reaction mixtures when the sample is resistant to release of nucleic acid at the LAMP reaction conditions. In an exemplary embodiment, the target polynucleotide resides inside a thermophile such as Thermus aquaticus, which thrives at temperatures at 70 degrees (Brock, 1969), and a skilled person will add lytic agents such as lysozyme to the LAMP reaction in order to have a successful reaction, A skilled person can add to the LAMP reaction lytic reagents compatible with a LAMP reaction, such as lysozyme, lyticase, zymolyase, and proteases to enhance release of nucleic acid from sample in addition to the release of nucleic acid from standard LAMP conditions even when not required.

In some embodiments, methods and systems herein described allow identifying a target microorganism or cell. In these embodiments, a skilled person identification can be performed by embodiments of methods and systems herein described in which one or more target polynucleotides amplified with LAMP reaction that are biomarkers for the microorganism. In some of those embodiments primers can be designed or otherwise selected to be specific to the biomarker of the target microorganism or cell. The target microorganism or cell can then be contacted with LAMP reaction mixture comprising a polymerase and the primers specific for the polynucleotide of the target at a time and conditions to allow amplification. The detecting of polynucleotide amplification can then be performed in accordance with any of the methods and techniques herein described. For example turbidity, colorimetry, or fluorescence can be used in conjunction with the LAMP reaction as a means to detect amplification of the target polynucleotide of the target cell. In some of these embodiments, one skilled in the art can use LAMP to discriminate between different states of a cell (see Examples 9 and 10). In some embodiments, the target microorganism or cell is a nonpathogenic cell. In other embodiments, the target microorganism or cell is a pathogenic cell.

As disclosed herein, the LAMP primers herein described can be provided as a part of systems to perform any assay, including any of the assays described herein. The systems can be provided in the form of kits of parts. In a kit of parts, the multi-ligand primers and other reagents to perform the assay can be comprised in the kit independently. The primers can be included in one or more compositions, and each primer can be in a composition together with a suitable vehicle.

Additional components can include labeled molecules and in particular, labeled polynucleotides, labeled antibodies, labels, microfluidic chip, reference standards, and additional components identifiable by a skilled person upon reading of the present disclosure. LAMP reagents, labeled molecules can be included in separate compositions as well as in prepared mixture wherein the reagents/molecules are included together with a suitable vehicle. The term “vehicle” as used herein indicates any of various media acting usually as solvents, carriers, binders or diluents for a reagent comprised in the composition as an active ingredient In some embodiments, detection of amplification can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Further advantages and characteristics of the present disclosure will become more apparent hereinafter from the following detailed disclosure by way of illustration only with reference to an experimental section.

### EXAMPLES

The methods and systems herein disclosed are further illustrated in the following examples, which are provided by way of illustration and are not intended to be limiting.

In particular, the following examples illustrate exemplary isothermal amplification of untreated samples and related methods and systems. In particular, the following examples illustrated isothermal amplification of pag (pX01), cap (pX02) and sap (chromosome) genes of Bacillus anthracis, from cells and/or spores, in cell cultures or other untreated samples. A person skilled in the art will appreciate the applicability and the necessary modifications to adapt the features described in detail in the present section, to additional target polynucleotide, viral particles, cells and in particular cellular microorganisms, solutions, methods and systems according to embodiments of the present disclosure.

The following materials and methods were used for all the methods and systems for the compositions methods and systems exemplified herein.

Bacterial Strains.

Bacterial strains used in this work are listed in TABLE 1. B. anthracis Sterne UT238 was originally provided as a gift from Dr. T. Koehler at the University of Texas to Dr. P. Jackson at LLNL. B. anthracis Sterne Dugway was obtained from the U.S. Department of Defense Proving Grounds at Dugway. B. globigii spores were a gift from Dr. Elizabeth Wheeler at LLNL and also were originally obtained from the U.S. Department of Defense Proving Grounds at Dugway. Pathogenic B. anthracis strains Ames, Vollum 1B and PAK-1 were obtained from the LLNL culture collection.

TABLE 1

| Strain | Source | pXO1 | pXO2 |
|---|---|---|---|
| B. globigii | Dugway Proving | − | − |
| | Grounds | | |
| B. anthracis Sterne UT238 | T. Koehler, UT | + | − |
| B. anthracis Sterne Dugway | Dugway Proving | + | − |
| | Grounds | | |
| B. anthracis Ames A0462 | LLNL | + | + |
| B. anthracis Vollum 1B A0488 | LLNL | + | + |
| B. anthracis PAK1 A0463 | LLNL | + | + |

Culture.

Liquid cultures of B. anthracis were grown in nutrient broth (NB) (EMD Chemicals/Merck KGaA, Damstadt, Germany). Nutrient agar cultures were inoculated from a 40% glycerol stock stored at −80° C. and incubated at 37° C. for 12-18 hours. An overnight culture was inoculated from a single colony picked from the nutrient agar and incubated at 37° C. for 12-18 hours in NB. A 1:50 dilution in NB was incubated to mid-log growth, OD600 of 0.45-0.9. Cultures were diluted in NB and plated for colony forming units on nutrient agar dishes.

Spore Preparation.

A mid-log culture of B. anthracis Sterne UT238 was inoculated onto 20 cm×20 cm surface of nutrient agar and allowed to absorb. Cells were then incubated for 3 days at 37° C. followed by 3-7 days at room temperature. Spores were harvested by scraping the agar surface into a 50 ml conical tube, resuspending in 30 ml MilliQ water, pelleting in an Eppendorf 5804 centrifuge (Eppendorf) with a fixed angle rotor at 6K RPM for 20 min followed by 3-6 washes in 30 ml MilliQ water. Spores were resuspended in a final volume of 5 ml water and stored at 4° C. Spore concentration was determined by CFU assay in triplicate. Spore quality and germination status was checked using brightfield, phase contrast and atomic force microscopy.

DNA Extraction.

DNA was extracted from overnight cultures of B. anthracis using the MasterPure® Gram positive DNA purification kit (Epicentre Biotechnologies, Madison, Wis.). DNA was quantified using a Qubit® 2.0 fluorometer (Invitrogen/Life Technologies, Carlsbad, Calif.) and confirmed via agarose gel electrophoresis.

LAMP.

The pag (pX01), cap (pX02) and sap (chromosome) genes of B. anthracis were amplified using primers previously described (Kurosaki et al., 2009). HPLC-purified primers were obtained from Biosearch Technologies (Biosearch Technologies, Novato, Calif.). Reactions contained 1.6 μM each FIP and BIP primers, 0.2 μM each F3 and B3 primers, 0.8 μM each LF and LB primers, 150 μM hydroxynaphthol blue (Sigma, St. Louis, Mo.), 1× ThermoPol reaction buffer (NEB, Ipswich, Mass.), 1.4 mM each dNTP (NEB), 0.8 M Betaine (Sigma), 8 mM MgSO4, 8 units Bst polymerase large fragment (NEB) and 5 μl sample brought up in a 25 μl final volume with nuclease-free water (NEB). Reactions containing protease inhibitors contained 1 μl Complete® mini-EDTA protease inhibitor cocktail (Roche Applied Science, Indianapolis, Ind.) prepared by resuspending 1 tablet in 10 ml nuclease-free water. Reactions were performed in a 96-well format and heated on an iCycler thermalcycler instrument (BioRad, Hercules, Calif.). Real-time LAMP reactions contained a 1×10⁻³ dilution of Picogreen (Invitrogen) in place of the HNB and were amplified on a CFX96 real-time instrument (BioRad).

DNase Treatment.

Twenty units of DNase 1 (NEB) was added to 50 pg purified DNA and 3×10⁶ CFU spores in DNase I buffer and incubated for 60 min at 37° C. followed by 20 min at 65° C. Resulting product was used directly in LAMP reaction.

### Example 1

### Colorimetric Detection of DNA Amplification from Unprocessed Bacillus anthracis Spores Using Loop-Mediated Isothermal Amplification

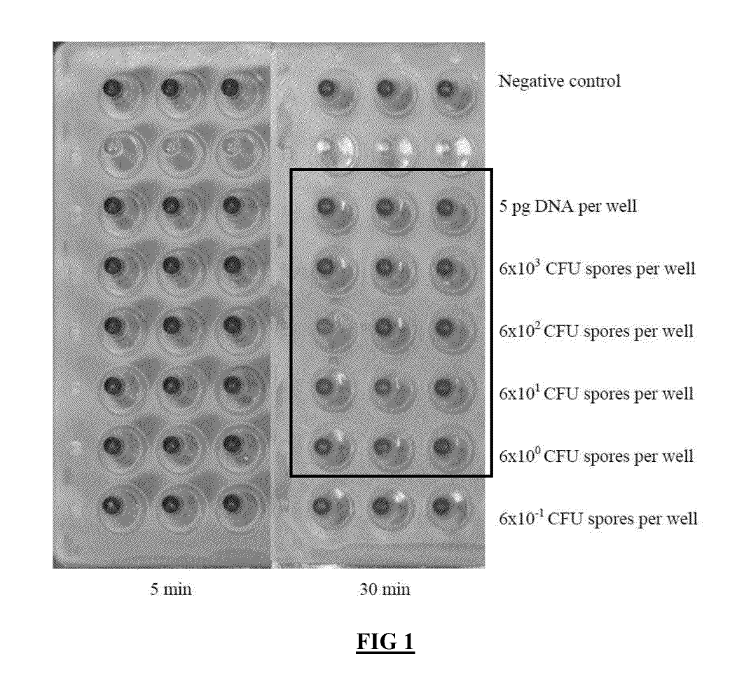

Spores or cells were added to the LAMP reaction mixture, heated to 63° C. and positive amplification was detected via color change from purple to blue as illustrated in FIG. 1. In addition, the inclusion of protease inhibitors in the LAMP reaction resulted in a ˜1-log increase in sensitivity. The addition of lytic enzymes, including lysozymes, muramidases or endolysins is expected to improve cell lysis and decrease time to positive amplification detection.

### Example 2

### Detection from Purified Nucleic Acid

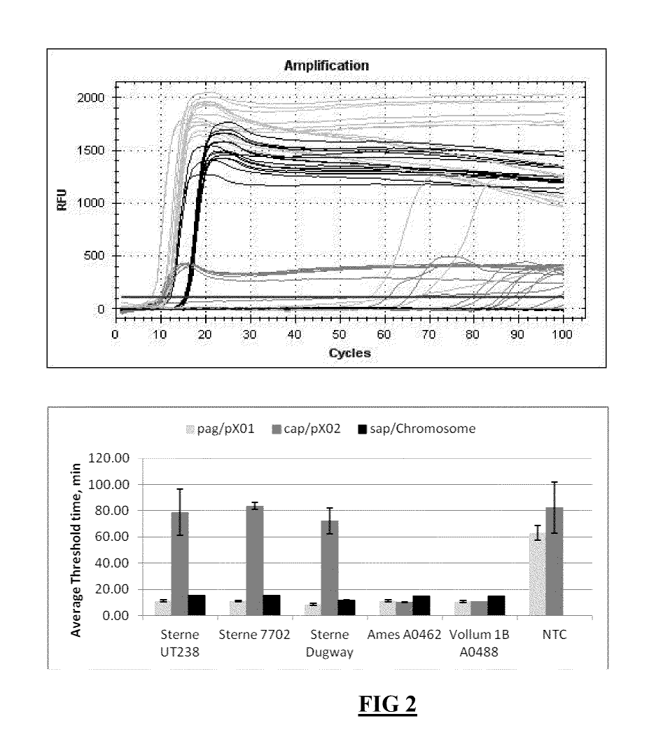

DNA purified from overnight cultures of B. anthracis was amplified on a BioRad CFX96 real-time PCR machine using picogreen and the FAM/SYBR-green detection channel. Other fluorescent dyes can be similarly used in conjunction with a detection channel that can accurately receive measurement as deemed appropriate by a skilled person. As shown in FIG. 2, DNA from three pX01+/pX02− Sterne isolates tested positive for the pX01 target pag and the chromosomal target sap, but were negative for the pX02 target cap. DNA from two pathogenic pX01+/pX02+ isolates, Ames and Vollum 1B tested positive for all three targets. These primer sets are therefore capable of discriminating between non-pathogenic and pathogenic isolates of B. anthracis using LAMP.

### Example 3

### Detection of Pag from Vegetative Cells

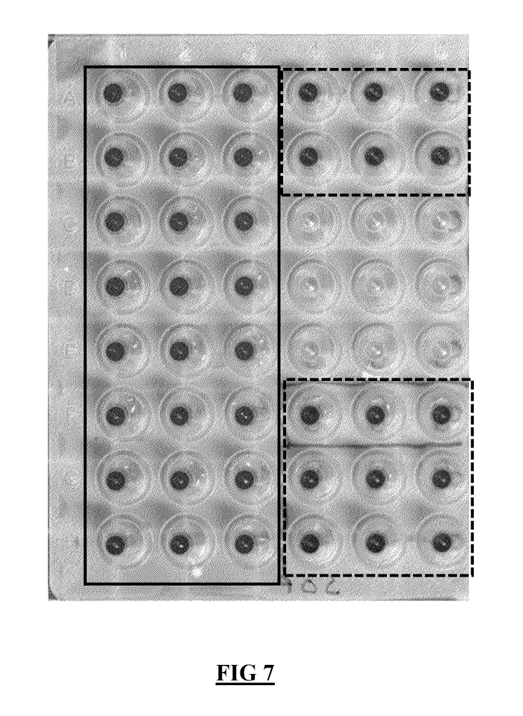

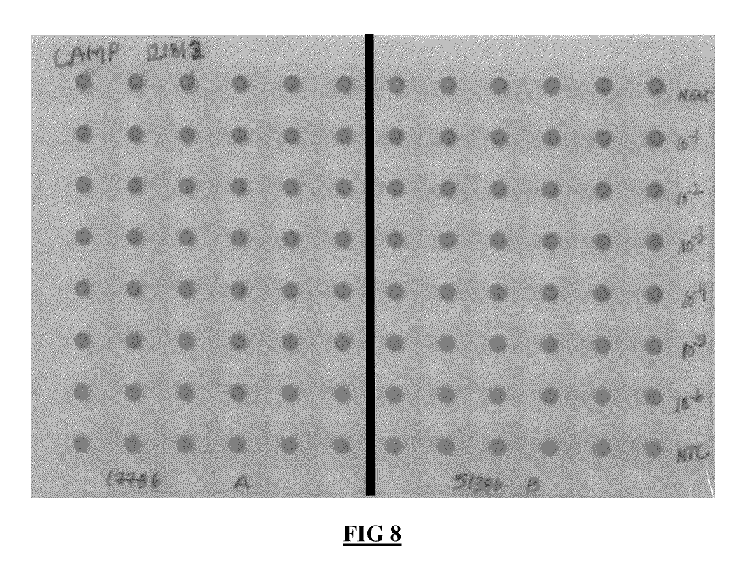

Exponentially growing B. anthracis Sterne cells either neat, or diluted in fresh media, were added directly to the LAMP reaction mixture and heated to 63° C. Color change was detected within 30 min for cells at concentrations as low as 30 CFU per reaction. FIG. 7 shows a mid-log culture of Bacillus anthracis cells and the LAMP primers targeting the protective antigen (pag) gene sequence for a 30 minute reaction. 30 CFU per reaction are wells G1, G2, and G3. Negative controls are in well F4-6, G4-6 and H4-6. All other wells are template positive beginning with 3×10⁵ CFU per reaction in wells A1-3 with a 10-fold dilution per row down to 3×10⁻¹ CFU per well in wells B4-6. Wells outlined by a black box indicates positive reaction, and wells outlined by a dashed box indicates negative reaction.

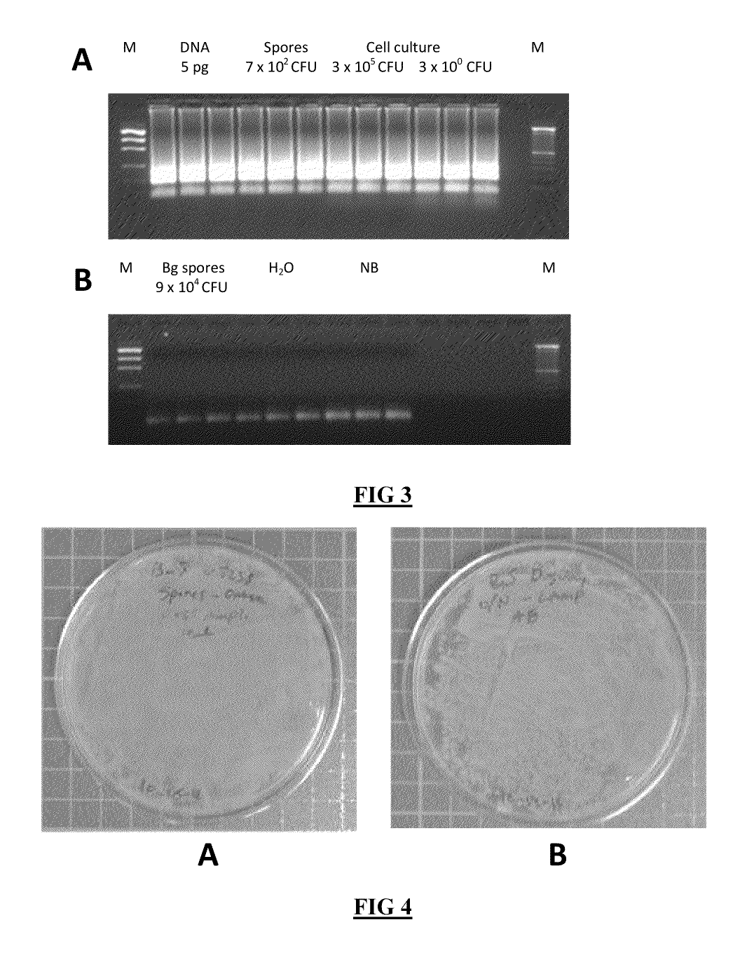

Due to the likely presence of proteases in the liquid culture (Haines 1931), a cocktail of protease inhibitors was tested to improve the sensitivity of the LAMP reaction on vegetative cells assayed directly from cell culture. The addition of protease inhibitors to the LAMP reaction lowered the detection limit by one log in these reactions from direct cell cultures as compared to similar reactions without protease inhibitors. Applicants' lower limit of detection corresponds to 3 CFU per reaction in 45 min compared to 30 CFU without protease inhibitors. To detect the lower limit, a dilution series of Bacillus anthracis cells were added to individual reaction tubes. The reaction tubes were then heated to 63° C. and checked for color change. At 45 min, 2 of 3 reactions containing 3 CFU cells and protease inhibitors were positive, while 0 of 3 reactions at this concentration without protease inhibitors were positive. FIG. 3 indicates that the pag LAMP product obtained from purified DNA, spores and cell culture is the same length.

### Example 4

### Detection of Pag from Agar Culture

Glycerol stocks of two B. anthracis Sterne isolates (UT238 and Dugway) were streaked onto nutrient agar and incubated overnight at 37° C. A loop of culture was harvested and resuspended in 0.5 ml fresh nutrient broth. Samples of each culture equating to 2×10⁵ CFU per reaction, were added directly to LAMP reaction mixture, heated to 63° C. and visualized for color change every 10 min. Color change was detected beginning around 20 min into the reaction and completed by 25 min. for both Sterne isolates.

### Example 5

### Detection of Pag from Overnight Culture

Overnight cultures initiated from single colonies of two B. anthracis Sterne isolates were added directly to LAMP reaction mixture, heated to 63° C. and visualized for color change every 10 min. Color change was detected beginning around 20 min into the reaction and completed by 25 min. for both Sterne isolates.

### Example 6

### Detection of Pag from Cells of Pathogenic Strains of B. Anthracis

Next, LAMP reactions were performed on cultures of three pathogenic strains of B. anthracis; Ames, Vollum 1B and PAK-1. Glycerol stocks were streaked on nutrient agar and incubated overnight at 36° C. A single colony was then inoculated into NB and incubated overnight at 36° C. with shaking A 1:50 dilution of overnight culture was made in fresh media and incubated at 36° C. with shaking to mid-log phase determined by OD₆₀₀ reading of ˜0.45. All culturing of pathogenic strains was performed under containment appropriate to RG3 organisms. Colonies from cultures grown on agar were picked into NB and added directly into LAMP reaction mixture. Additionally, overnight liquid cultures and mid-log cultures were added directly into the LAMP reaction mixture. Positive amplification of the pag target was detected by color change within 20 min for all strains and cultures tested.

### Example 7

### Detection of Pag from Washed Spore Preparations

Freshly prepared B. anthracis Sterne spores and suspensions of stored spores (4 weeks at 4° C.) were loaded directly into a LAMP reaction solution and heated. Visual color change (violet to sky blue) was observed for positive samples within 20-25 min, whereas B. globigii control spores produced no color change in the LAMP reaction. Concentrations as low as <10 CFU per 25 μl reaction were consistently detected in under 45 min. To determine if exogenous DNA may be influencing the amplification, spores were incubated in DNase 1 prior to running the LAMP reaction. DNase-treated spores had a slightly slower amplification time requiring an additional 5 min for colorimetric detection at the lower limit of detection of ≦10 CFU per reaction when compared to untreated spores. This suggests the presence of free DNA in the spore preparation can influence the onset of observed color change. Control DNA treated with DNase 1 did not amplify as determined by a lack of color change within 60 min at 63° C.

### Example 8

### Cell Culture of Samples Following LAMP

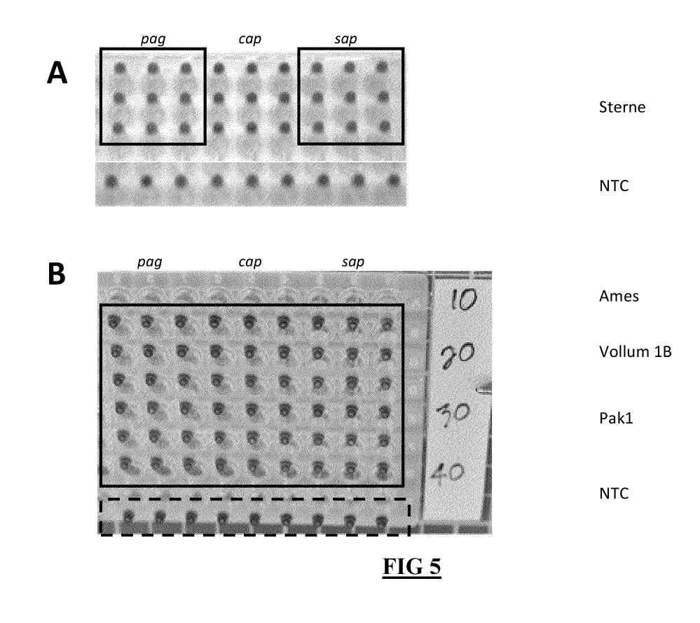

Bacillus spores are heat resistant and can be induced to germinate by incubating at temperatures from 60-100° C. Isolation of Bacillus spp. from environmental samples often involves heat shock treatment to kill off vegetative cells in the sample. In the laboratory, heat shock treatment for 20 min at 65° C. is used to eliminate vegetative cells from spore preparations (Hill 1949). Since this temperature is nearly identical to the optimal LAMP reaction temperature, whether samples containing spores of B. anthracis are viable following the LAMP reaction was examined. To determine whether spores would survive, germinate and outgrow following positive amplification, 10 μl of post-LAMP reaction containing B. anthracis Sterne spores were plated onto nutrient agar and incubated overnight. As shown in FIG. 4, a lawn of B. anthracis Sterne was present confirming spore survivability during nucleic acid amplification. A follow-on colony-forming assay was performed using pre- and post-LAMP samples and results indicated 100% recovery of spores following LAMP. Combined, these results indicate that additional avenues of sample analysis and confirmation, such as cell culture, are possible following the initial colorimetric detection step.

### Example 9

### Discrimination of Non-Pathogenic from Pathogenic B. Anthracis Cells or Spores Via LAMP

LAMP results using purified DNA as template indicated that the use of the three primer sets (pag, cap, sap) could discriminate non-pathogenic from pathogenic strains. Therefore, we tested whether this was also true using cells or spores as template. Spores from two non-pathogenic strains (Sterne UT238 and Dugway) and cells from three pathogenic strains (Ames, Vollum and PAK1) were added directly to LAMP reaction mixtures targeting, pag, cap and sap. As shown in FIG. 5, non-pathogenic pX01+/pX02− Sterne cells were positive for pag and sap within 25 min of initial heating, while the pathogenic pX01+/pX02+ Ames, Vollum 1B and PAK-1 strain cells were positive for all three targets within the same time frame. These results indicate that in under 30 minutes a sample of spores or cells can be assayed and determined to be a non-pathogenic pX01+/pX02− form of B. anthracis or a pathogenic pX01+/pX02+ form of B. anthracis.

### Example 10

### Discrimination of Non-Pathogenic from Pathogenic B. Anthracis Cells Via LAMP

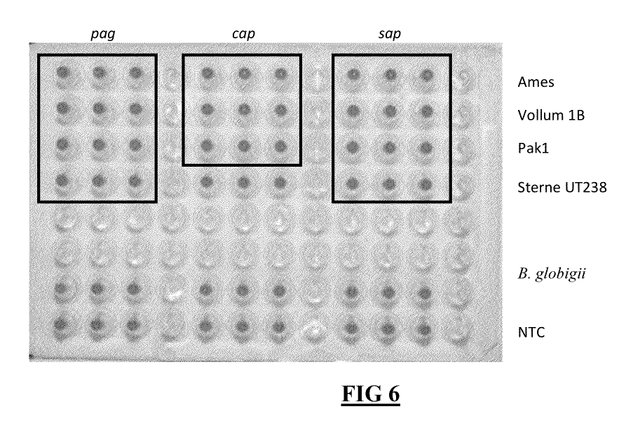

LAMP results using purified DNA as template indicated that the use of the three primer sets (pag, cap, sap) could discriminate non-pathogenic from pathogenic strains. Therefore, we tested whether this was also true using cells as template. Liquid cultures from one non-pathogenic strain, Sterne UT238 and three pathogenic strains (Ames, Vollum and PAK1) were added directly to LAMP reaction mixtures targeting, pag, cap and sap along with a negative control culture of B. globigii. As shown in FIG. 6, non-pathogenic pX01⁺/pX02⁻ Sterne cells were positive for pag and sap within 25 min of initial heating, while the pathogenic pX01⁺/pX02⁺ Ames, Vollum 1B and PAK1 strain cells were positive for all three targets within the same time frame. B. globigii cells were negative for all three targets. These results indicate that in 30 minutes a sample of cells can be assayed and determined to be a non-pathogenic pX01⁺/pX02⁻ form of B. anthracis or a pathogenic pX01⁺/pX02⁻ form of B. anthracis.

Published results indicate that LAMP reactions are less susceptible to inhibitors of PCR reactions, such as urine, decanted feces, whole blood and blood culture media (Francois 2011). Applicants' results indicate that direct testing of aqueous suspensions of B. anthracis spores and vegetative cells in solid and liquid culture using the LAMP assay combined with colorimetric detection will greatly simplify and shorten the detection process by eliminating nucleic acid purification. Freshly prepared spores, partially germinated spores, colonies from agar dishes, overnight cultures, cells of early- and mid-log cultures of the Sterne strain along with overnight cultures, cells of early- and mid-log cultures of three pathogenic strains of B. anthracis have been successfully amplified following this protocol. Additionally, protease inhibitors added to the LAMP reaction may improve lower limits of detection. Finally, testing samples with a combination of primer sets for targets on both B. anthracis plasmids and the chromosome may permit identification of non-pathogenic and pathogenic B. anthracis within 30 minutes, while allowing for the direct re-culturing of positive samples for additional testing and verification.

### Example 11

### Detection of Toxin a and Toxin B from C. botulinum Cells Via LAMP

Various C. botulinum strains were grown anaerobically in liquid growth medium or on solid agar. Cells in growth medium were diluted in a 10-fold dilution series and added directly to LAMP reaction mixtures containing primers published in Sakuma et al., J Applied Microbiology 106 (2009) p. 1252-1259 targeting the Toxin A (BoNT/A) or Toxin B (BoNT/B) genes of C. botulinum. Cells grown on agar were scraped off the agar, resuspended in 1 ml growth medium, diluted in a 10-fold dilution series and added to the LAMP reaction mixture. Reactions were heated to 63° C. and visualized for color change.

Cultures grown in liquid growth medium were detected at 30 min at concentrations of ˜100 CFU per reaction and at 60 min at concentrations of ˜10 CFU per reaction. Cultures grown on agar were detected at 30 min at concentrations of 1000 CFU per reaction and at 60 min at concentrations of ˜100 CFU per reaction. Results at 60 min are shown in FIG. 8. ATCC strain 17786 encodes the Toxin A (BoNT/A), while ATCC strain 51386 encodes the Toxin B (BoNT/B) gene. 10-fold dilution series is shown on the right-hand side and negative controls occupy each well of the bottom row. Columns 1-3 and 7-9 represent triplicate samples of cultures grown on agar, while columns 2-4 and 10-12 represent cultures grown in liquid growth medium.

The examples set forth above are provided to give those of ordinary skill in the art a complete disclosure and description of how to make and use the embodiments of the compositions, arrangements, devices, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

Bearinger, J. P., L. C. Dugan, B. R. Baker, S. B. Hall, K. Ebert, V. Mioulet, M. Madi, and D. P. King. 2011. Development and initial results of a low cost, disposable, Point-of-Care testing device for pathogen detection. IEEE Trans. Biomed. Eng. 58:805-808.
Brittan, H. G. 1978. The use of hydroxynaphthol blue in the ultramicro-determination of alkaline earth and lanthanide elements: an improved method. Analyt. Chim. Acta. 96:165-170.
Cardosa, T. C., H. F. Ferrar, L. C. Bregano, C. Silva-Frade, A. C. G. Rosa, and A. L. Andrade. 2010. Visual detection of turkey coronavirus RNA in tissues and feces by reverse-transcription loop-mediated isothermal amplification (RT-LAMP) with Hydroxynaphthol blue dye. Mol Cell Probes. 24:415-417.
Dukes, J. P., D. P. King, and S. Alexanderson. 2006. Novel reverse transcription loop-mediated isothermal amplification for rapid detection of foot-and-mouth disease virus. Arch. Virol. 151:1093-1106.
Francois, P., M. Tangomo, J. Hibbs, E-J. Bonetti, C. C. Boehme, T. Notomi, M. D. Perkins, and J. Schrenzel. 2011 Robustness of a loop-mediated isothermal amplification reaction for diagnostic applications. FEMS Immunol. Med. Microbiol. 62:41-48.
Goto, M., E. Honda, A. Ogura, A. Nomoto, and K.-I. Hanaki. 2009. Colorimetric detection of loop-mediated isothermal amplification reaction by using hydroxynaphthol blue. Biotech. 46:167-172.
Haines, R. B. 1931. CXCIX. The formation of bacterial proteases, especially in synthetic media. Biochem. J. 25:1851-1859.
Hatano, B., T. Maki, T. Obara, H. Fukumoto, K. Hagisawa, Y. Matsushita, A. Okutani, B., Bazartseren, S. Inoue, T. Sata, and H. Katano. 2010. LAMP using a disposable pocket warmer for Anthrax detection, a highly mobile and reliable method for anti-bioterrorism. Jpn. J. Infect. Dis. 63:36-40.
Hill, G. M. 1949. Chemical factors in the germination of spore-bearing aerobes. The effect of yeast extract on the germination of Bacillus anthracis and its replacement by adenosine. Biochem. J. 45:353-362.
Inglesby, T. M., D. A. Henderson, J. G. Bartlett, M. S. Ascher, E. Eitzen, A. M. Friedlander, J. Jauer, J. McDade, et al., 1999. Anthrax as a Biological Weapon. Medical and Public Health Management. J. Americ. Med. Assoc. 281:1735-1745.
Jain, N., J. S. Kumar, M. M., Parida, S. Merwyn, G. P. Rai, and G. S. Agarwal. 2011. Real-time loop-mediated isothermal amplification assay for rapid and sensitive detection of anthrax spores in spiked soil and talcum powder. World J. Microbiol. Biotechnol. 27:1407-1413.
Jernigan, J. A., D. S. Stephens, D. A. Ashford, C. Omenaca, M. S. Topiel, M. Galbraith, M. Tapper, T. L. Fisk, et al., 2001. Bioterrorism-related inhalational Anthrax: The first 10 cases reported in the United States. Emer. Infect. Dis. 7:933-944.
Keim, P., K. Smith, C. Keys, H. Takahashi, T. Kurata, and A. Kaufman. 2001. Molecular investigation of the Aum Shinrykio anthrax release in Kameido, Japan. J. Clin. Microbiol. 39:4566-4567.
Kuboki, N. N. Inoue, T. Sakurai, F. Di Cello, D. J. Grab, H. Suzuki, C. Sugimoto, and I. Igarashi. Loop-mediated isothermal amplification for detection of African trypanosomes. J. Clin. Microbiol. 41:5517-5524.
Kurosaki, Y., T. Sakuma, A. Fukuma, K. Kawamoto, N. Kamo, S.-l. Makin, and J. Yasuda. 2009. A simple and sensitive method for detection of Bacillus anthracis by loop-mediated isothermal amplification. J. Appl. Microbiol. 107:1947-1956.
Luo, L., K. Nie, M-J. Yang, M. Wang, J. Li, C. Zhang, H-T. Liu, and X-J. Ma. 2011. Visual detection of high-risk human papillomavirus genotypes 16, 18, 45, 52 and 58 by loop-mediated isothermal amplification with hydroxynaphthol blue dye. J. Clin. Microb. 49:3545-3550.
Ma, X-j, Y-l Shu, K. Nie, M. Qin, D-y. Wang, R-b. Gao, M. Wang, L-y. Wen, F. Han, S-m. Zhou, X. Zhao, Y-h. Cheng, D-x. Li, X-p. Dong. 2009. Visual detection of pandemic influenza A H1N1 Virus 2009 by reverse-transcription loop-mediated isothermal amplification with Hydroxynaphthol blue dye. J. Virol. Methods. 167:214-217.
Mikesell, P., B. E. Ivins, J. D. Ristroph, and T. M. Dreier. 1983. Evidence for plasmid-mediated toxin production in Bacillus anthracis. Infect Immun. 39:371-376.
Nagamine, K., T. Hase, and T. Notomi. 2002. Accelerated reaction by loop-mediated isothermal amplification using loop primers. Mol. Cell. Probes 16:223-229.
Nie, K., Y. Zhang, L. Luo, M-J. Yang, X-M. Hu, M. Wang, S-L. Zhu, F. Han, W-b. Xu, and X-j Ma. 2011. Visual detection of human enterovirus 71 subgenotype C4 and Coxsackievirus A16 by reverse transcription loop-mediated isothermal amplification with the Hydroxynaphthol blue dye. J. Virol. Meth. 175:283-286.
Notomi, T., H. Okayama, H. Masubuchi, T. Yonekawa, K. Watanabe, N. Amino, and T. Hase. 2000. Loop-mediated isothermal amplification of DNA. Nucleic Acids Res. 28:E63.
Ohtsuka, K., K. Yanagawa, K. Takatori, and Y. Hara-Kudo. 2005. Detection of Salmonella enterica in naturally contaminated liquid eggs by loop-mediated isothermal amplification, and characterization of Salmonella isolates. Appl. Environ. Microbiol. 71:6730-6735.
Okinaka, R. T., K. Cloud, O. Hampton, A. R. Hoffmaster, K. K. Hill, P. Keim, T. M. Koehler,
G. Lamke, et al., 1999. Sequence and organization of pX01, the large Bacillus anthracis plasmid harboring the Anthrax toxin genes. J. Bacteriol. 181:6509-6515.
Parida, M., S. Sannarangaiah, P. K. Dash, P. V. L. Rao, and K. Morito. 2008. Loop mediated isothermal amplification (LAMP): a new generation of innovative gene amplification technique; perspectives in clinical diagnosis of infectious diseases. Rev. Med. Virol. 18:407-421.
Pile, J. C., J. D. Malone, E. M. Eitzen, and A. M. Friedlander. 1998. Anthrax as a potential biological warfare agent. Arch. Int. Med. 158:429-434.
Qiao Y.-M., Y.-C. Guo, X.-E. Zhang, Y.-F. Zhou, Z.-P. Zhang, H.-P. Wei, R.-F. Yang, and D.-B. Wang. 2007. Loop-mediated isothermal amplification for rapid detection of Bacillus anthracis spores. Biotech. Lett. 29:1939-1946.
Read, T. D., S. N. Peterson, N. Tourasse, L. W. Baillie, I. T. Paulsen, K. E. Nelson, H. Tettelin, D. E. Fouts, et al., 2003. The genome sequence of Bacillus anthracis Ames and comparison to closely related bacteria. Nature. 423:81-86.
Sakuma, T., Kurosaki, Y., Fujinami, Y., Takizawa, T. and Yasuda, J. (2009), Rapid and simple detection of Clostridium botulinum types A and B by loop-mediated isothermal amplification. Journal of Applied Microbiology, 106: 1252-1259.
Tanner, Nathan A., Yinhua Zhang, and Thomas C. Evans Jr. “Simultaneous multiple target detection in real-time loop-mediated isothermal amplification.” BioTechniques 53 (2012): 81-89.
Wastling, S. L., K. Picozzi, A. S. L. Kakembo, and S. C. Welburn. 2010. LAMP for human African trypanosomiasis: A comparative study of detection formats. PLoS Negl. Trop. Dis. 4:e865.
Mattingly, Stephen J., and Gary K. Best. “The effect of temperature on lysis of cells and cell walls of Bacillus psychrophilus.” Canadian journal of microbiology 17.9 (1971): 1161-1168.
Membrillo-Hernández, Jorge, et al. “Thermally-induced cell lysis in Escherichia coli K12.” Journal of basic microbiology 35.1 (1995): 41-46.
Ray, P. H., and T. D. Brock. “Thermal lysis of bacterial membranes and its prevention by polyamines.” Journal of General Microbiology 66.2 (1971): 133-135.
Ogata, S., and M. Hongo. “Lysis induced by sodium ion and its relation to lytic enzyme systems in Clostridium saccharoperbutylacetonicum.” Journal of general microbiology 81.2 (1974): 315-323.
Brock, Thomas D., and Hudson Freeze. “Thermus aquaticus gen. n. and sp. n., a nonsporulating extreme thermophile.” Journal of Bacteriology 98.1 (1969): 289-297.

1. A method to detect a target polynucleotide in an untreated sample, the method comprising performing loop-mediated isothermal amplification on the untreated sample with primers specific for the target polynucleotide; and detecting amplification of the target polynucleotide following the performing.

2. The method of claim 1, wherein the target polynucleotide is comprised in the untreated sample at a concentration lower than about 10 fg.

3. The method of claim 1, further comprising performing an additional assay to detect the target polynucleotide.

4. The method of claim 1, wherein the untreated sample further includes polynucleotides other than the target polynucleotide.

5. The method of claim 1, wherein the loop mediated isothermal amplification is performed in presence of a lytic enzyme or other reagents suitable to increase availability of the target polynucleotide.

6. The method of claim 1, wherein the target polynucleotide is comprised within a cell.

7. The method of claim 6, wherein the cell is a cellular microorganism.

8. The method of claim 7, wherein the cellular microorganism is Bacillus anthracis.

9. The method of claim 6, wherein the target polynucleotide is specific for the cell.

10. A system for detection of a target polynucleotide in an untreated sample, the system comprising primers specific for the target polynucleotide and reagents for performing loop-mediated isothermal amplification for simultaneous combined or sequential use in detecting target polynucleotide in an untreated sample.

11. The system of claim 10, wherein the primers and the reagents are for simultaneous, combined or sequential use is performed according to the method of any one of claim 1.

12. A method to identify a target cell, the method comprising contacting the target cell with a polymerase and primers specific for a target polynucleotide specific for the target cell for a time under condition to allow performance of loop-mediated isothermal amplification; and detecting polynucleotide amplification following the contacting.

13. The method of claim 12, wherein the cell is a cellular microorganism.

14. The method of claim 13, wherein the cellular microorganism is Bacillus anthracis.

15. The method of claim 12, the method further comprising performing an additional assay for identifying the target cell following the detecting.

16. A system for identifying a target cell in an untreated sample, the system comprising primers specific for the target cell and reagents for performing loop-mediated isothermal amplification for simultaneous combined or sequential use in detecting target cell in an untreated sample.

17. The system of claim 16, wherein the primers and the reagents are for simultaneous, combined or sequential use is performed according to a method to identify a target cell, the method comprising contacting the target cell with a polymerase and primers specific for a target polynucleotide specific for the target cell for a time under condition to allow performance of loop-mediated isothermal amplification; and detecting polynucleotide amplification following the contacting.

18. A method to identify Bacillus anthracis in an untreated sample, the method comprising contacting the untreated sample with a polymerase and primers specific for the Bacillus anthracis for a time under condition to allow performance of loop-mediated isothermal amplification; and detecting polynucleotide amplification following the contacting.

19. The method of claim 18, further comprising treating the sample to extract a target polynucleotide following the detecting; the target polynucleotide specific for Bacillus anthracis; and detecting the target polynucleotide following the treating.

20. A system for detection of a Bacillus anthracis in an untreated sample, the system comprising primers specific for Bacillus anthracis and reagents for performing loop-mediated isothermal amplification for simultaneous combined or sequential use in detecting Bacillus anthracis in an untreated sample.

21. The system of claim 20, further comprising reagents for extracting a polynucleotide according to claim 19.

22. The system of claim 20, wherein the primers and the reagents are for simultaneous, combined or sequential use is performed according to a method to identify Bacillus anthracis in an untreated sample, the method comprising contacting the untreated sample with a polymerase and primers specific for the Bacillus anthracis for a time under condition to allow performance of loop-mediated isothermal amplification; and detecting polynucleotide amplification following the contacting.
